(19) United States
(12) Patent Application Publication
(10) Pub. No.: US 2014/0039473 A1
(43) Pub. Date: Feb. 6, 2014

(54) Dermatological Treatment Device With Real-Time Energy Control

(71) Applicant: TRIA Beauty, Inc., Dublin, CA (US)

(72) Inventors: Harvey I-Heng Liu, Fremont, CA (US); John P. Beale, Mountain View, CA (US)

(73) Assignee: TRIA Beauty, Inc., Dublin, CA (US)

(21) Appl. No.: 13/895,245

(22) Filed: May 15, 2013

Related U.S. Application Data
(60) Provisional application No. 61/647,249, filed on May 15, 2012.

(51) Int. Cl. A61B 18/20

(52) U.S. Cl. CPC A61B 18/20
USPC 606/9

(57) ABSTRACT

A dermatological treatment device may include a device body; a radiation source housed by the device body and configured to emit pulses of treatment light for delivery to the skin to provide a dermatological treatment; a photo detector housed by the device body and configured to detect light from the skin; and control electronics housed by the device body and configured to: during the delivery of a particular treatment light pulse from the radiation source, receive signals from the photo detector; during the delivery of the particular treatment light pulse, automatically calculate at least one pulse parameter value for the particular treatment light pulse based at least on the signals received from the photo detector; and automatically control at least one parameter of the particular treatment light pulse based on the at least one calculated pulse parameter value for the particular treatment light pulse.

### CROSS-REFERENCE TO RELATED APPLICATIONS

This application claims the benefit of U.S. Provisional Application No. 61/647,249 filed on May 15, 2012, which disclosure is herein incorporated by reference in its entirety.

### TECHNICAL FIELD

The present disclosure is related to dermatological treatment devices that include a real-time energy control, e.g., to analyze and control energy pulses in real time.

### BACKGROUND

Radiation-based treatment of tissue is used for a variety of applications, such as hair removal, skin rejuvenation, wrinkle treatment, acne treatment, treatment of vascular lesions (e.g., spider veins, diffuse redness, etc.), treatment of cellulite, treatment of pigmented legions (e.g., age spots, sun spots, moles, etc.), tattoo removal, and various other treatments. For example, some treatments use a laser source to deliver laser radiation to an area of tissue on a person's body, e.g., the skin or internal tissue, to treat the tissue in a photochemical, photobiological, thermal, or other manner, which can be ablative or non-ablative, among other properties, depending on the particular application.

Some laser-based treatment devices apply laser radiation directly from the laser source to the target tissue to create a pattern of radiated areas (e.g., spots, lines, or other shapes) in the tissue. Others include optics between the laser source and the target tissue. Such optics may include optical elements such as lenses, mirrors, and other reflective and/or transmissive elements, for controlling optical parameters of the beam, such as the direction, shape (e.g., convergent, divergent, collimated), spot size, angular distribution, temporal and spatial coherence, and/or intensity profile of the beam. Some devices include systems for scanning a laser beam in order to create a pattern of radiated areas (e.g., spots, lines, or other shapes) in the tissue. For some applications, the scanned pattern of radiated areas overlap each other, or substantially abut each other, or are continuous, in order to provide generally complete coverage of a target area of tissue. For other applications, e.g., certain wrinkle treatments and other skin rejuvenation treatments, the scanned radiated areas may be spaced apart from each other such that only a fraction of the overall target area of the tissue is radiated. In this case, there are generally regions of untreated tissue between regions of treated tissue. This latter type of treatment is known as “fractional” treatment (or more specifically, fractional photothermolysis) because only a fraction of the target area is irradiated.

Laser-based treatment devices may deliver radiation as continuous wave (CW) radiation, manually pulsed radiation, automatically pulsed radiation, or in any other manner, and according to any suitable parameters, e.g., wavelength, current, power level, etc. For example, a wavelength of about 650 nm to about 1100 nm (e.g., about 810 in some applications) may be used for hair removal treatment. As another example, wavelengths absorbed by water in the skin, e.g., between 1400 nm and 2000 nm, may be used for certain treatments. For certain “fractional” skin treatments, a wavelength of about 1450-1550 nm±50 nm may be used, with a total energy of about 2 mJ-30 mJ delivered to the target tissue at each treatment zone, or “microthermal zone” (MTZ).

### SUMMARY

Embodiments of the present disclosure provide devices and methods to automatically control the amount of radiation energy (e.g., light energy) applied to a region of the skin for a dermatological treatment, e.g., to cause a rise in the temperature of tissue chromophores by a specified amount. Some embodiments provide dermatological treatment devices including a real time energy control system configured to provide real time control of radiation delivered to the skin, e.g., by controlling the delivered radiation during an individual radiation pulse or otherwise in real time.

One example embodiment provides a dermatological treatment device including a device body; a radiation source housed by the device body and configured to emit pulses of treatment light for delivery to the skin to provide a dermatological treatment; a photo detector housed by the device body and configured to detect light from the skin; and control electronics housed by the device body and configured to: during the delivery of a particular treatment light pulse from the radiation source, receive signals from the photo detector; during the delivery of the particular treatment light pulse, automatically calculate at least one pulse parameter value for the particular treatment light pulse based at least on the signals received from the photo detector; and automatically control at least one parameter of the particular treatment light pulse based on the at least one calculated pulse parameter value for the particular treatment light pulse.

Another example embodiment provides a method of automated control of a dermatological treatment device, which includes: operating a radiation source to emit treatment light pulses for delivery to the skin to provide a dermatological treatment; detecting light from the skin using a photo detector; during the delivery of a particular treatment light pulse from the radiation source, receiving signals from the photo detector; during the delivery of the particular treatment light pulse, automatically calculating at least one pulse parameter value for the particular treatment light pulse based at least on the signals received from the photo detector; and automatically controlling at least one parameter of the particular treatment light pulse based on the at least one calculated pulse parameter value for the particular treatment light pulse.

### BRIEF DESCRIPTION OF THE DRAWINGS

Some embodiments of the disclosure may be understood by referring, in part, to the following description and the accompanying drawings wherein:

FIG. 1 illustrates components of an example dermatological treatment device including a real-time energy control system, according to certain embodiments;

FIG. 2 illustrates one example embodiment of the dermatological treatment device of FIG. 1;

FIG. 3 illustrates components of an example real-time energy control system, according to one embodiment;

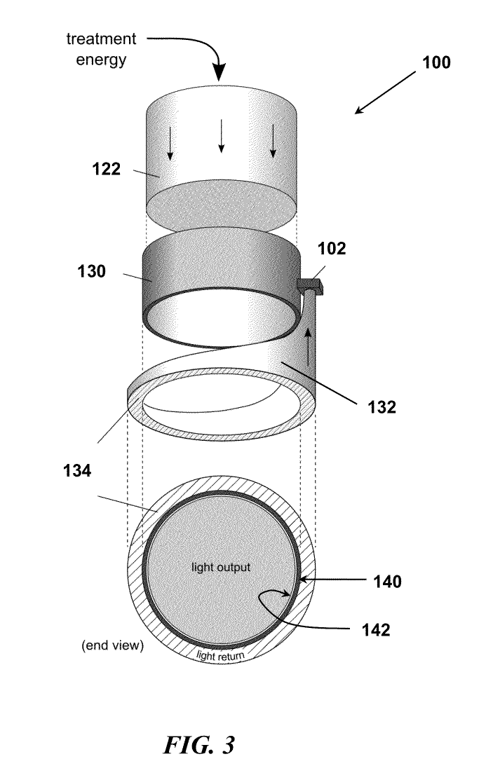

FIG. 4 illustrates aspects of real-time energy control for different skin types or pigmentation levels, according to certain embodiments;

FIG. 5 illustrates example treatment control sequences implemented by a treatment device including a real-time energy control system, for different skin types or pigmentation levels, according to an example embodiment; and

FIG. 6 illustrates an example method of providing real-time energy control during a dermatological treatment, according to an example embodiment.

### DETAILED DESCRIPTION OF THE DRAWINGS

Some embodiments provide dermatological treatment devices including a real time energy control system configured to provide real time control of radiation delivered to the skin. Such dermatological treatment devices may be configured for providing any suitable radiation-based dermatological treatments, e.g., by causing a rise in the temperature of tissue chromophores by a specified amount. Example radiation-based dermatological treatments include skin resurfacing, skin rejuvenation, wrinkle treatment, removal or reduction of pigmentation, hair removal, acne treatment, skin tightening, redness, vascular treatments such as telangectasia or port-wine stains, stretch marks, anti-aging, or anti-inflammatory skin treatments such as treating rosacea, acne, or vitiligo. Other embodiments may apply to non-skin tissue treatment, such as eye tissue or internal organs.

Some embodiment provide a hand-held compact device including a laser radiation source for providing laser-based dermatological treatments, and a real time energy control system for controlling aspects of the laser-based radiation in real time, e.g., during an individual laser pulse or otherwise in real time.

The dermatological treatment device may provide radiation at any suitable wavelength for providing any suitable treatment, e.g., any of the treatments listed above. For example, dermatological treatment device may provide radiation at a wavelength of 808 nm which is absorbed by melanin in the epidermis, 1440-1460 nm to target water absorption in the dermis, 1720-1760 nm for absorption in sebaceous glands, and/or any other wavelengths for these or other types of dermatological treatments.

The radiation energy may be generated, for example, by one or more diode lasers. The intention of the treatment is to cause some desired change in the skin which is accomplished by absorption of a specific amount of energy within the target area of skin, for example around hair follicles to inhibit hair growth (e.g., laser hair removal), or in the dermis to promote collagen production (e.g., fractional photothermolysis). It may be desirable to deliver an appropriate energy dose to the target, such that the energy dose is neither too small, and thus lacking the desired treatments effects, nor too large, which might cause damage or undesired effects in the skin. The appropriate energy dose may in general depend on the density and absorptivity of the target chromophores in the skin, which may vary between individuals and between different areas on the same individual. Thus, some treatment devices disclosed herein are configured to automatically adjust the applied energy dose as required for each treatment area.

In some embodiments, the device is configured to provide real time energy control of the amount of radiation energy applied to the skin. For the purposes of this disclosure, “real time” operations are automated operations performed within or substantially within the time required for completion of such operations by one or more automated electronic components, including time required for electronic data collection and/or electronic data processing, without significant delays unrelated to the automated completion of such operations.

Thus, “real time energy control” means collecting electronic sensor signals (e.g., regarding the skin or treatment radiation) during delivery of the radiation to the skin, analyzing the collected sensor signals by a digital processor, and controlling one or more operational parameters of the radiation delivery to the skin based on the analysis of the sensor signals by the digital processor, within or substantially within the required time for collecting and processing the electronic sensor signals by the relevant sensor(s) and digital processor, without introducing significant delays unrelated to such functions.

In some embodiments, real time energy control provides a control response time (i.e., time from collection of relevant sensor data to operational adjustment of the treatment device (e.g., the radiation source)) of less than 10 milliseconds. In some embodiments, real time energy control provides a control response time of less than 5 milliseconds. In some embodiments, real time energy control provides a control response time of less than 1 millisecond. In some embodiments, real time energy control provides a control response time of less than 300 microseconds.

Devices that provide pulse radiation delivery (e.g., for hair removal, fractional treatment, etc.) may provide “real time pulse control,” which means collecting electronic sensor signals (e.g., regarding the skin or treatment radiation) during delivery of a particular radiation pulse to the skin, analyzing the collected sensor signals by a digital processor during the particular radiation pulse, and controlling one or more operational parameters of the radiation delivery during the particular radiation pulse (e.g., controlling the duration or end time of the pulse, or providing mid-pulse (i.e., intra-pulse) adjustment of the power level, radiation intensity, fluence, or any other parameters of the radiation pulse. Some devices may be configured to take multiple sensor measurements during each pulse in order to provide such real time pulse control. The device may be configured to take such sensor measurements at any suitable frequency, e.g., approximately: every 250 μs, every 500 μs, every 1 ms, every 2 ms, every 5 ms, every 10 ms, every 20 ms, every 50 ms, every 100 ms, or any other suitable frequency.

Real time pulse control may be provided for treatments or configurations having a wide variety of pulse duration. For example, fractional treatment devices having a typical pulse duration of less than 20 milliseconds may provide real time pulse control to (a) control the duration or end time of each pulse and/or (b) provide one or more mid-pulse adjustments of one or more pulse parameters. Example fractional treatment devices having a typical pulse duration of between 1 ms and 10 ms may provide real time pulse control to (a) control the duration or end time of each pulse and/or (b) provide a series of mid-pulse adjustments (where appropriate) of one or more pulse parameters during each pulse, at any suitable mid-pulse adjustment frequency (e.g., approximately: every 500 μs, every 1 ms, every 2 ms, or any other suitable frequency). Example fractional treatment devices having a typical pulse duration of less than 1 ms may provide real time pulse control to (a) control the duration or end time of each pulse and/or (b) provide at least one mid-pulse adjustments (where appropriate) of one or more pulse parameters during each pulse, at any suitable mid-pulse adjustment frequency (e.g., approximately: every 500 μs, every 250 μs, or faster).

As another example, hair removal devices having a typical pulse duration of between 10 and 500 milliseconds may provide real time pulse control to (a) control the duration or end time of each pulse and/or (b) provide one or more mid-pulse adjustments of one or more pulse parameters. Example fractional treatment devices having a typical pulse duration of 100-500 ms may provide real time pulse control to (a) control the duration or end time of each pulse and/or (b) provide a series of mid-pulse adjustments (where appropriate) of one or more pulse parameters during each pulse, at any suitable mid-pulse adjustment frequency (e.g., approximately: every 10 ms, every 50 ms, every 100 ms, or any other suitable frequency). Example fractional treatment devices having a typical pulse duration of less than 100 ms may provide real time pulse control to (a) control the duration or end time of each pulse and/or (b) provide at least one mid-pulse adjustments (where appropriate) of one or more pulse parameters during each pulse, at any suitable mid-pulse adjustment frequency (e.g., approximately: every 5 ms, every 10 ms, every 20 ms, or any other suitable frequency).

In some embodiments, systems and methods disclosed herein may provide real time energy control to measure and adjust the amount of treatment energy applied to a treatment area so as to raise the superficial skin temperature by a specified amount. At least two treatment modes are considered: a gliding treatment mode and a stamping treatment mode. In the gliding treatment mode, the real time energy control may take into account the speed or displacement of the device gliding along the skin surface, as measured by a velocity sensor or a displacement sensor. If displacement occurs during a pulse, then the area exposed to light is larger than if the device is stationary, and the device may compensate for such movement by controlling the radiation source accordingly. In the stamping treatment mode, the treatment may be applied to one localized area of the skin at a time.

In some embodiments, measured diffuse-scattered light is used to calculate absorbed energy within tissue in real time (e.g., with a control response time less than 10 ms, less than 5 ms, less than 1 ms, or less than every 300 μs), in order to automatically control treatment energy in real time to deliver the desired amount of energy to accomplish a particular treatment objective.

In some example embodiments, the device is a compact-hand-held device for providing laser-based hair removal by providing pulsed or continuous wave (“CW”) radiation as the device is moved across the skin, e.g., in a gliding mode or a stamping mode. In other example embodiments, the device is a compact-hand-held device for providing laser-based fractional treatment (e.g., non-ablative fractional treatment) by pulsing one or more lasers as the device is moved across the skin.

As discussed above, in some embodiments, the device provides pulsed energy beams to the skin to provide a fractional dermatological treatment, e.g., skin resurfacing, skin rejuvenation, wrinkle treatment, removal or reduction of pigmentation, treatment of coarse skin caused by photodamage, etc. Each pulsed energy beam forms an irradiated treatment spot (or “treatment spot”) on the surface of the skin, and a three-dimensional volume of thermally damaged (or otherwise influenced, such as photochemically) skin extending below the surface of the skin, referred to herein as a micro thermal zone (MTZ). Each MTZ may extend from the skin surface downward into the skin, or may begin at some depth below the skin surface and extend further downward into the skin, depending on the embodiment, device settings, or particular application. The device may be configured to generate an array of MTZs in the skin that are laterally spaced apart from each other by volumes of untreated (i.e., non-irradiated or less irradiated) skin. For example, an application end of the device (also referred to herein as the device “tip”) may be manually moved (e.g., in a sliding manner) across the surface of the skin during a treatment session. An energy beam or beams may be pulsed (to generate MTZs in the skin) during the movement of the device across the skin (referred to herein as a “gliding mode” treatment), or between movements of the device to different locations on the skin (referred to herein as a “stamping mode” treatment), or a combination of these modes or different modes. The skin's healing response, promoted by the areas of untreated (i.e., non-irradiated) skin between adjacent MTZs, provides fractional treatment benefits in the treatment area (e.g., skin resurfacing or rejuvenation, wrinkle removal or reduction, pigment removal or reduction, etc.). In some embodiments or applications, the compact, hand-held device may yield results similar to professional devices, but leverages a home use model to more gradually deliver the equivalent of a single professional dose over multiple treatments or days. Skin rejuvenation generally includes at least one or more of treatments for wrinkles, dyschromia, pigmented lesions, actinic kerotosis, melasma, skin texture, redness or erythema, skin tightening, skin laxity, and other treatments.

As used herein, “fractional” treatment means treatment in which individual treatment spots generated on the skin surface are physically separated from each other by areas of non-irradiated (or less irradiated) skin (such that the MTZs corresponding to such treatment spots are generally physically separated from each other). In other words, in a fractional treatment, adjacent treatment spots (and thus their corresponding MTZs) do not touch or overlap each other. In some embodiments in which one or more radiation sources are pulsed to generate a successive series of treatment spots on the skin, the pulse rate may be set or selected based on a typical or expected speed at which the device is manually moved or “glided” across the skin, referred to herein as the “manual glide speed” (e.g., in a gliding mode operation of the device). In particular, the pulse rate may be set or selected such that for a range of typical or expected manual (or mechanically-driven) glide speeds, adjacent treatment spots are generally physically separated from each other by areas of non-treated skin (i.e., fractional treatment is provided). In some embodiments, the pulse rate may be set or selected such that for a range of typical or expected manual glide speeds, adjacent treatment spots are physically separated from each other from a predetermined minimum non-zero distance, e.g., 500 μm. For example, in some embodiment, a pulse rate of between 2 and 30 HZ (e.g., about 15 Hz) may be selected for providing a desired fractional treatment for typical or expected manual glide speeds of between 1 and 6 cm/sec.

In some embodiments, the device may be controlled to prevent, limit, or reduce the incidence or likelihood of treatment spot overlap, e.g., based on feedback from one or more sensors (e.g., one or more dwell sensors, motion/speed sensors, and/or displacement sensors). For example, the device may monitor the speed or displacement of the device relative to the skin and control the radiation source(s) accordingly, e.g., by turning off the radiation source, reducing the pulse rate, etc. upon detecting that the device has not been displaced on the skin a minimum threshold distance from a prior treatment location. Further, in some embodiments, the pulse rate may be automatically adjustable by the device and/or manually adjustable by the user, e.g., to accommodate different manual glide speeds and/or different comfort levels or pain tolerance levels of the user.

In some embodiments, the device is configured to be manually scanned across the skin, rather than using an automated scanning system (e.g., including systems for moving optical elements and/or the laser or other radiation source) present in various existing devices. In some embodiments the device does not include any moving optics (or any optics at all, as discussed below). In some embodiments, both the radiation source and the radiation path(s) from the radiation source to the skin are fixed with respect to the device housing.

Further, the device may be configured for “direct exposure” or “indirect exposure” radiation, and/or for “close proximity” or “remote proximity” radiation, depending on the particular embodiment and/or configuration of the device. “Direct exposure” embodiments or configurations do not include any optics downstream of the radiation source for affecting or treating the beam(s) generated by the radiation source (the term “optics” is defined below in this document). Some direct exposure devices may include a window (e.g., to protect the radiation source and/or other internal components of the device) that does not substantially affect the beam. A window may be formed from any suitable material, e.g., sapphire, quartz, diamond, or other material transparent at the frequency of the radiation source 14 and may also have a good thermal coefficient.

Thus, embodiments of the device may create a desired pattern of MTZs without using microlenses or other similar optics. Thus, embodiments of the device may provide increased optical efficiency, reduced power requirements, simpler and less expensive manufacturing, increased compactness, and/or enhanced reliability as compared with certain non-ablative fractional treatment devices that use microlenses or other similar optics for creating MTZ arrays. However, it should be understood that certain embodiments of the device may include one or more optics, e.g., for desired beam shaping.

In contrast, “indirect exposure” embodiments or configurations include one or more optics downstream of the radiation source for affecting or treating the beam(s) generated by the radiation source. Optics may allow the radiation source to be positioned at any desired distance from the application end of the device that contacts the skin during treatment (and thus at any desired distance from the target surface) or to affect other radiation properties.

In “close proximity” embodiments or configurations, the emitting surface of the radiation source is positioned within 10 mm of the skin-contacting surface of the device (i.e., the leading surface of the device tip), such that the emitting surface of the radiation source is positioned within 10 mm of the skin surface when the device tip is positioned in contact with the skin. As discussed below, this distance is referred to herein as the “proximity gap spacing.” In contrast, in “remote proximity” embodiments or configurations, the proximity gap spacing (between the emitting surface of the radiation source and the skin-contacting surface of the device) is greater than 10 mm. Some close proximity embodiments, due to the small proximity gap spacing and thus short travel distance of the beam(s) from the radiation source to the skin, may omit precision-aligned optics (or all optics) that may be needed in similar remote proximity embodiments, thus providing a direct exposure, close proximity configuration. Some particular embodiments discussed below include a radiation source configured for direct exposure and close proximity radiation, wherein the emitting surface of the radiation source is positioned within 10 mm of the skin surface, with no optics (e.g., only a window, open space, protective coating, or similar feature) between the radiation source and the skin. Direct exposure, close proximity embodiments may be particularly compact. Some direct exposure, close proximity embodiments may provide a high optical throughput and may be capable of generating relatively high-power emissions in a compact battery-operated device.

It should be understood that “direct exposure” is not synonymous with “close proximity,” and likewise “indirect exposure” is not synonymous with “remote proximity.” That is, direct exposure embodiments or configurations may be configured for either close proximity or remote proximity radiation, depending on the particular embodiment or configuration. Similarly, indirect exposure embodiments or configurations may be configured for either close proximity or remote proximity radiation, depending on the particular embodiment or configuration. For example, some embodiments may include a very small lens (e.g., a cylindrical or ball lens) downstream of the light source, but wherein the emitting surface of the radiation source is still within 10 mm of the skin surface during treatment.

In some embodiments, the radiation engine, real-time energy control system, and radiation delivery components (if any) of the device have an all-solid-state construction that excludes any automated or mechanically moving parts for dynamically moving the radiation source or the direction or location of the propagated beam(s) relative to the device housing, e.g., including (a) any motorized or otherwise moving beam-scanning elements, such as motorized or otherwise moving optical elements to scan a beam to multiple different directions or locations relative to the device housing (e.g., galvo-controlled mirrors or rotating multi-faceted scanning elements), and (b) any motorized or other elements for physically moving the radiation source and any associated beam delivery elements (e.g., a laser, LED, fiber, waveguide, etc.). Such embodiments may reduce noise, increase the reliability of the device, reduce manufacturing cost and complexity, and/or increase compactness of the finished device with low or minimal component count.

In some embodiments, the device has an all-solid-state construction with no automated moving parts at all, including no automated or mechanically moving parts for dynamically moving the radiation source or direction and location of the propagated radiation beam(s) relative to the device housing (as discussed above), no automated moving components of the real-time energy control system, no fans, other motors, or other automated moving parts.

Certain example embodiments are handheld, battery powered, compact skin treatment devices with all solid-state components, configured to provide direct exposure and/or close-proximity radiation, and for providing skin area coverage via manual scanning of the device across the surface of the skin, in a gliding or stamping mode operation, and using CW or pulsed radiation.

In some embodiments, the device is fully or substantially self-contained in a compact, hand-held housing. For example, in some battery-powered embodiments of the device, the radiation source, real-time energy control system, user interface(s), control electronics, sensor(s), battery or batteries, fan(s) or other cooling system (if any), and/or any optics (if any), are all contained in a compact, hand-held housing. Similarly, in some wall-outlet-powered embodiments of the device, the radiation source, real-time energy control system, user interface(s), control electronics, sensor(s), battery or batteries, fan(s) or other cooling system (if any), and/or any optics (if any), are all contained in a compact, hand-held housing, with only the power cord extending from the device.

In other embodiments, one or more main components of the device may be separate from the device housing, and connected by any suitable physical or wireless means (e.g., wire, cable, fiber, wireless communications link, etc.)

In some embodiments, the device provides eye safe radiation, e.g., due to the divergence of the beam(s) delivered by the radiation source and/or using particular optics (e.g., a mixer and/or diffuser) and/or using an eye safety control system including one or more sensors, and/or by any other suitable manner. In some laser-based embodiments or settings, the device meets the Class 1M or better (such as Class 1) eye safety classification per the IEC 60825-1. In other laser-based embodiments or settings, the device falls outside the IEC 60825-1 Class 1M eye safety classification by less than 25% of the difference to the next classification threshold. In still other laser-based embodiments or settings, the device falls outside the IEC 60825-1 Class 1M eye safety classification by less than 50% of the difference to the next classification threshold.

In some embodiments, the device is eye safe, hand held, manufacturable without excessive labor costs, requires low power consumption, and effective. In some embodiments, the device eliminates the need for optical scanners, microlenses, or other complex optical and mechanical devices, for creating multiple MTZs in the skin. In particular embodiments, the device is battery powered, with a single, fixed location, repetitively-pulsed edge emitting laser diode for creating an array of MTZs in the skin by manually scanning the device across the skin while the radiation source is repetitively pulsed, with each pulse creating either a single MTZ or multiple MTZs in the skin, depending on the configuration of the radiation source.

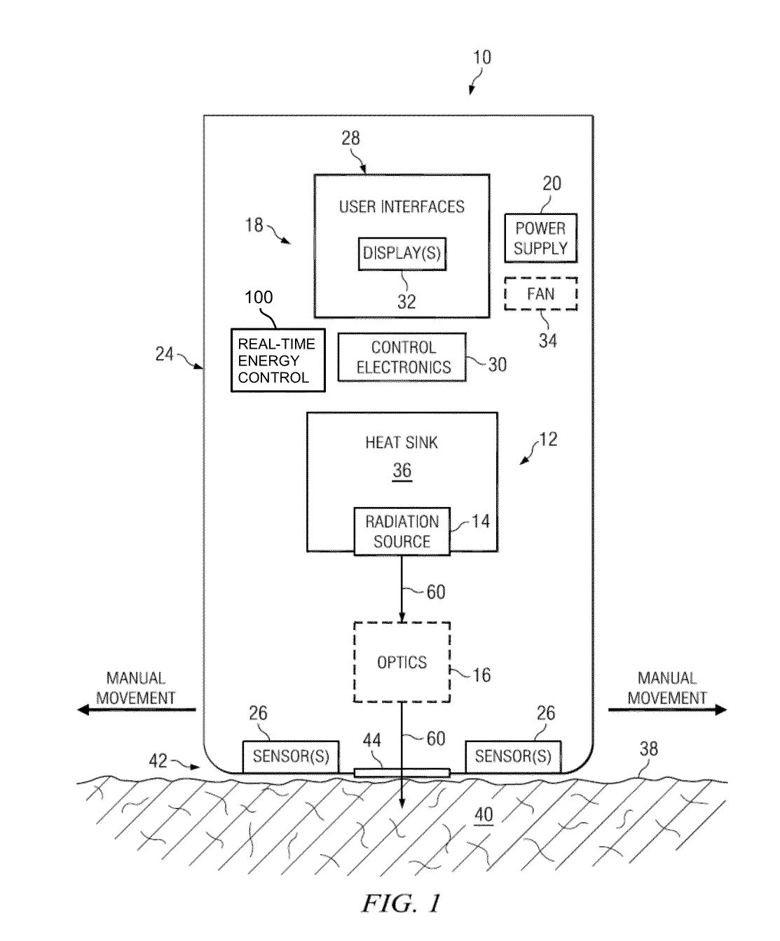

FIG. 1 illustrates components of an example treatment device 10, according to certain embodiments. Treatment device 10 may include a laser engine 12 including a treatment radiation source 14 configured to generate treatment radiation (e.g., laser radiation), in the form or one or more radiation beams 60, optics 16 for delivering the treatment radiation to a target area 40 (e.g., an area of tissue), control systems 18, one or more power supplies 20, one or more fans 34, and one or more detectors or sensors 26. Control systems 18 include a real-time energy control system 100, which incorporates one or more detectors or sensors 26 and other suitable electronics for providing real-time control of operations aspect(s) of device 10, as discussed below.

As discussed below, “direct exposure” embodiments may omit optics 16 such that no optics are provided between radiation source 14 and the target surface, for direct exposure of the target tissue. In some direct exposure embodiments, radiation source is located in close proximity to the target skin surface (e.g., less than 10 mm, less than 2 mm, or even less than 1 mm from the target skin surface).

The components of device 10 may be provided in a structure or housing 24, or alternatively may be provided in separate structures or housings and connected in any suitable manner, e.g., via fiber optic or other cabling. Housing 24 may define an application end (or “treatment tip”) 42 configured to be placed in contact with the target surface (e.g., skin) during treatment of the target area 40. Application end 42 may include or house various user interfaces, including the treatment delivery interface for delivering laser radiation to the user and/or one or more sensors 26 for detecting various characteristics of the target surface and/or treatment delivered by device 10.

In some embodiments, application end 42 may include an aperture or window 44 through which the treatment radiation, in the form or one or more beams 60, is delivered to the target surface, or alternatively, an optical element 16 (e.g., a lens) may be located at application end 42 and configured for direct contact or very close proximity with the skin during treatment. In some embodiments, one or more sensors 26 (including at least one detector or sensor of real-time energy control system 100) may also be arranged to operate through window 44 (e.g., by delivering radiation to, and/or receiving radiation from, the skin via window 44).

Device 10 may include any other components suitable for providing any of the functionality discussed herein or other related functionality known to one of ordinary skill in the art.

Radiation source 14 may comprise any one or more type of device configured to radiate energy, e.g., in the form of one or more beams, to produce one or more irradiated areas on the skin that provide a dermatological treatment. As used herein, “radiation” may include any radiative energy, including electromagnetic radiation, UV, visible, and IP light, radio frequency, ultrasound, microwave, etc. A radiation source may include any suitable device for radiating one or more coherent or incoherent energy beams, e.g., a laser, LED, flashlamp, ultrasound device, RF device, microwave emitter, etc. In some embodiments, the radiation source is a laser, e.g., an edge emitting laser diode, laser diode bar, fiber laser, HeNe laser, YAG laser, VCSEL laser, or other types of laser, that generates one or more laser beams delivered to the skin to effect a treatment.

Further, radiation source 14 may be configured for and/or operated at any suitable wavelength to provide the desired treatment. For example, radiation source 14 may be configured for and/or operated at a wavelength of about 810 nm (e.g., 810 nm ±30 nm) for providing hair removal treatment. As used herein, the term “hair removal” encompasses both removal of hair and inhibition of hair growth/regrowth. As another example, radiation source 14 may be configured for and/or operated at a wavelength that is absorbed by water in the skin, e.g., between 1400 nm and 2000 nm, e.g., for certain photothermolysis treatments. In some embodiments, radiation source 14 may be configured for and/or operated at a wavelength of between 1400 nm and 1550 nm, e.g., for acne treatment or certain fractional non-ablative skin treatments. In other embodiments, radiation source 14 may be configured for and/or operated at a wavelength of between 1700 nm and 1800 nm, e.g., for sebaceous gland related treatment like acne.

In still other embodiments, radiation source 14 may be configured for and/or operated at a wavelength of between 1900 nm and 1950 nm, e.g., for pigmented lesion treatment like solar lentigo.

Further, radiation source 14 may be configured or operated to deliver continuous wave (CW) radiation, pulsed radiation, or in any other manner. In some embodiments, device 10 controls radiation source 14 to provide CW radiation, e.g., for using device 10 in a gliding mode to provide bulk heating skin tightening, hair removal, or acne treatment. In other embodiments, device 10 controls radiation source 14 to provide manually pulsed radiation, e.g., for using device 10 in a stamping mode to provide hair removal. In still other embodiments, device 10 controls radiation source 14 to provide automatically pulsed radiation, e.g., for using device 10 in a gliding mode to provide selective photothermalysis. For example, in some embodiments, device 10 may be configured to sequentially deliver a series of laser beams to the target area 40, while being manipulated by the user in a stamping mode or in a gliding mode, to generate treatment zones (e.g., continuous or discontinuous line segments) that are spaced apart from each other by areas of non-irradiated skin between the adjacent treatment zones, to provide a fractional treatment to the tissue, e.g., for skin rejuvenation, wrinkle treatment, or treatment of pigmented legions (e.g., age spots, sun spots, moles, etc.).

Certain embodiments of device 10 include one or more optics 16 downstream of radiation source 14 for directing or treating the radiation emitted from radiation source 14 before reaching the target surface. Optics 16 may allow for radiation source 14 to be positioned at any desired distance from the application end 42 of the device that contacts the skin during treatment (and thus at any desired distance from the target surface). Embodiments of device 10 that include optics 16 downstream of laser engine 12 are referred to herein as “indirect exposure” embodiments.

Optics 16 may include any number and types of optical elements, e.g., lenses, mirrors, and other reflective and/or fully or partially transmissive elements, for delivering the radiation generated by laser engine 12, in the form of one or more beams, to the target area 40 and, if desired, for treating the one or more beams, such as adjusting the treatment zone size, intensity, treatment zone location, angular distribution, coherence, etc.

As used herein, an “optic” or “optical element” may mean any element that deflects a radiation beam, influences the angular distribution profile (e.g., angle of convergence, divergence, or collimation) of a radiation beam in at least one axis, influences the focus of the beam in at least one axis, or otherwise affects a property of the radiation. Thus, optics include mirrors and other reflective surfaces, lenses, prisms, light guides, gratings, filters, etc. For the purposes of this disclosure, optics do not generally include planar or substantially planar transmissive elements such as transmissive windows or films, such as those that serve as transmissive aperture that protect internal components.

Other embodiments of device 10 do not include any optics 16 downstream of radiation source 14. Such embodiments are referred to herein as “direct exposure” embodiments. A “direct exposure” embodiment or configuration does not include any optics downstream of the radiation source 14 for affecting or treating the beam(s) generated by radiation source 14. Some direct exposure devices may include a window (e.g., to protect the radiation source 14, sensor(s) 26, and/or other internal components of the device) that does not substantially affect the radiation beam(s). A window may be formed from any suitable material, e.g., sapphire, quartz, diamond, or other material transparent at the frequency of the radiation source 14 and preferably also having a good thermal coefficient.

In some embodiments, radiation source 14 may be positioned very close to the application end 42 of the device that contacts the skin during treatment (and thus very close to the target surface). For example, some direct exposure devices are also configured for “close proximity” radiation, in which the radiation source 14 are positioned such that the emitting surface is less than 10 mm from the leading surface of the application end 42 (and thus less than 10 mm from the target surface when the application end 42 is placed in contact with the skin). In some embodiments, the radiation source 14 are positioned such that the emitting surface is less than 2 mm from the leading surface of the application end 42/less than 2 mm from the target surface. In particular embodiments, the radiation source 14 are positioned such that the emitting surface is less than 1 mm from the leading surface of the application end 42/less than 1 mm from the target surface. Still further, in some embodiments, the radiation source 14 are positioned such that the emitting surface is less than 500 μm, 200 μm, or even 100 μm from the leading surface of the application end 42 or the target surface.

Control systems 18 may be configured to control one or more components of device 10 (e.g., laser engine 12 and/or a beam scanning system 142). Control systems 18 may include, for example, any one or more of the following: a laser control system for controlling aspects of the generation and delivery of radiation to the user; a displacement-based control system for controlling aspects of device 10 based on the determined displacement of device 10 across to the skin (e.g., as device is moved across the skin during treatment in a gliding mode or stamping mode), e.g., relative to a prior treatment position; a temperature control system; an eye safety control system to help prevent exposure of the eyes (e.g., the corneas) to the treatment radiation (an eye safety control system may be omitted in embodiments in which the laser radiation emitted from device 10 is inherently eye-safe); and/or a battery/power control system.

In addition, as shown in FIG. 1, control systems 18 include a real-time energy control system 100, which incorporates one or more detectors or sensors 26 and other suitable electronics for providing real-time control of operations aspect(s) of device 10, as discussed below.

Control systems 18 may include one or more sensors 26, user interfaces 28 for facilitating user interaction with device 10, and control electronics 30 for processing data (e.g., from sensors 26 and/or user interfaces 28) and generating control signals for controlling various components of device 10. Control electronics 30 may include one or more memory devices and processors for storing and executing logic instructions or algorithms or other data. Memory devices may include any one or more tangible, non-transitory device for storing electronic data (including logic instructions or algorithms), such as any type of RAM, ROM, Flash memory, or any other suitable volatile and/or non-volatile memory devices. Logic instructions or algorithms may be implemented as software, firmware, or any combination thereof. Processors may include any one or more devices, e.g., one or more microprocessors and/or microcontrollers, for executing logic instructions or algorithms to perform at least the various functions of device 10 discussed herein. Control electronics 30 may include exclusively analog electronics or any combination of analog and digital electronics.

Control systems 18, including real-time energy control system 100, may control components or aspects of device 10 based on feedback from sensors 26, user input received via user interfaces 28, and/or logic instructions/algorithms. For example, in some embodiments, control system 18 may control the operation of radiation source 14 based at least on feedback from a displacement sensor for detecting the displacement of device 10 relative to the skin 40 as the device is moved across the skin. Thus, for example, control system 18 may control radiation source 14 based on signals from a displacement sensor indicating that device 10 has moved a certain distance across target area 40 from a prior treatment position. As another example, control system 18 may control the operation of radiation source 14 based at least on feedback from a glide speed sensor for detecting the speed of device 10 moving across the skin. Thus, for example, control system 18 may control radiation source 14 based on signals from a glide speed sensor indicating that device 10 is moving at a particular speed across the skin 40.

More specifically, control system 18 may be configured to control one or more operational parameters of device 10. For example, control system 18 may control the treatment level (e.g., low power level, medium power level, or high power level) or treatment mode (e.g., gliding mode vs. stamping mode; or manually pulsed mode vs. automatically pulsed mode; or rapid-pulse mode vs. slow-pulse mode; or initial treatment mode vs. subsequent treatment mode; etc.), the performance of radiation source 14 (e.g., on/off, pulse-on time, pulse-off time, pulse duty cycle, pulse frequency, temporal pulse pattern, etc.), parameters of the radiation (e.g., radiation wavelength, intensity, power, fluence, etc.), the configuration or operation of one or more optical elements (e.g., the operation of a beam scanning system having rotating or otherwise moving optics or other elements), and/or any other aspects of device 10.

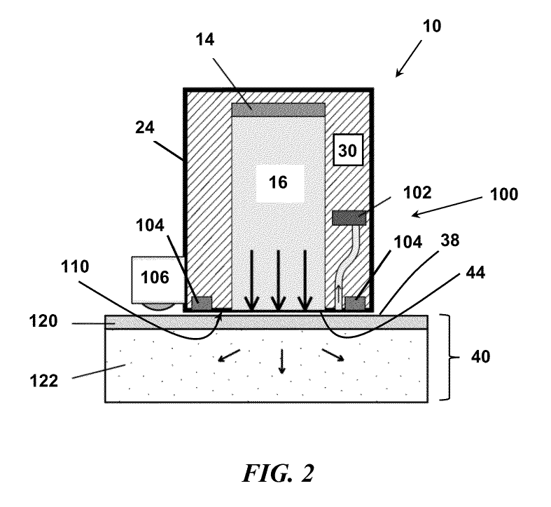

Sensors 26 may include any one or more sensors or sensor systems for sensing or detecting data regarding device 10, the user, the operating environment, or any other relevant parameters. For example, sensors 26 may include one or more photodetector (e.g., photodetector 102 shown in FIG. 2) for use by real-time energy control system 100. In some embodiments, the photodetector(s) are configured to detect treatment radiation from radiation source 14 that is received-and-remitted by the skin (e.g., after laterally scattering in the epidermis), thereby providing an indication of the amount of the treatment radiation that is absorbed in the underlying skin tissue.

Sensors 26 may also include one or more of the following types of sensors: (a) one or more displacement sensor for determining the displacement of device 10 relative to the skin as device 10 is moved (e.g., glided) across the skin, (b) one or more glide speed sensor for determining the speed, rate, or velocity of device 10 moving (e.g., gliding) across the skin, (c) one or more skin-contact sensor for detecting proper contact between device 10 and the skin, (d) one or more pressure sensor for detecting the pressure of device 10 pressed against the skin, (e) one or more temperature sensor for detecting the temperature of the skin, a region of the skin, and/or components of device 10, (f) one or more radiation sensor for detecting one or more parameters of radiation (e.g., intensity, fluence, wavelength, etc.) delivered to the skin, (g) one or more color/pigment sensor for detecting the color or level of pigmentation in the skin, (h) one or more treatment endpoint sensor, e.g., a color/pigment sensor, for detecting an influence of the radiation on the skin (e.g., erythema, temperature, perifollicular edema, etc.) during or after a treatment, (i) one or more eye safety sensor for preventing unwanted eye exposure to light from radiation source 14, (j) one or more dwell sensor for detecting if the device is stationary or essentially stationary with respect to the skin, (k) one or more roller-type sensors for detecting the displacement and/or glide speed of device 10, and/or any other suitable types of sensors.

In some embodiments, control systems 18 may include any of the various sensors and/or control systems disclosed in U.S. Ser. No. 13/366,246, in addition to real-time energy control system 100. For example, with reference to U.S. Ser. No. 13/366,246, control system 18 may include one or more displacement sensor, motion/speed sensor, skin-contact sensor, pressure (or force) sensor, temperature sensor, radiation sensor, color/pigment sensor, eye safety sensor, dwell sensor, and/or roller-based sensor, as disclosed in U.S. Ser. No. 13/366,246. As another example, again with reference to U.S. Ser. No. 13/366,246, control systems 18 may include any or all of a radiation source control system, a displacement-based control system, a user interface control system, a temperature control system, and/or a battery/power control system.

User interfaces 28 may include any systems for facilitating user interaction with device 10. For example, user interfaces 28 may include buttons, switches, knobs, sliders, touch screens, keypads, devices for providing vibrations or other tactile feedback, speakers for providing audible instructions, beeps, or other audible tones; or any other methods for receiving commands, settings, or other input from a user and providing information or output to the user. User interfaces 28 may also include one or more displays 32, one or more of which may be touchscreens for receiving user input. One or more user interfaces 28 or portions thereof may be included in a separate housing from the treatment device, such as in a smart charging dock or a personal computer, and the treatment device may communicate with the separate housing via hardwire (such as a cable or jack), wireless methods (such as infrared signals, radio signals, or Bluetooth), or other suitable communication methods.

Power supplies 20 may include any one or more types and instances of power supplies or power sources for generating or supplying power to the various components of device 10. For example, power supplies 20 may comprise one or more rechargeable or non-rechargeable batteries, capacitors, super-capacitors, DC/DC adapters, AC/DC adapters, and/or connections for receiving power from an outlet (e.g., 110V wall outlet). In some embodiments, power supplies 20 include one or more rechargeable or non-rechargeable batteries, e.g., one or more Li containing cells or one or more A, AA, AAA, C, D, prismatic, or 9V rechargeable or non-rechargeable cells.

### Real-Time Energy Control System

As discussed above, real-time energy control system 100 is configured to provide real-time control of radiation delivered to the skin, e.g., by controlling the delivered radiation during an individual radiation pulse or otherwise in real time. For the purposes of this disclosure, “real time” operations are automated operations performed within or substantially within the time required for completion of such operations by one or more automated electronic components, including time required for electronic data collection and/or electronic data processing, without significant delays unrelated to the automated completion of such operations.

Thus, “real time energy control” means collecting electronic sensor signals (e.g., regarding the skin or treatment radiation) during delivery of the radiation to the skin, analyzing the collected sensor signals by a digital processor, and controlling one or more operational parameters of the radiation delivery to the skin based on the analysis of the sensor signals by the digital processor, within or substantially within the required time for collecting and processing the electronic sensor signals by the relevant sensor(s) and digital processor, without introducing significant delays unrelated to such functions.

In some embodiments, real time energy control provides a control response time (i.e., time from collection of relevant sensor data to operational adjustment of the treatment device (e.g., the radiation source)) of less than 10 milliseconds. In some embodiments, real time energy control provides a control response time of less than 5 milliseconds. In some embodiments, real time energy control provides a control response time of less than 1 millisecond. In some embodiments, real time energy control provides a control response time of less than 300 microseconds.

Devices that provide pulse radiation delivery (e.g., for hair removal, fractional treatment, etc.) may provide “real time pulse control,” which means collecting electronic sensor signals (e.g., regarding the skin or treatment radiation) during delivery of a particular radiation pulse to the skin, analyzing the collected sensor signals by a digital processor during the particular radiation pulse, and controlling one or more operational parameters of the radiation delivery during the particular radiation pulse (e.g., controlling the duration or end time of the pulse, or providing mid-pulse (i.e., intra-pulse) adjustment of the power level, radiation intensity, fluence, or any other parameters of the radiation pulse. Some devices may be configured to take multiple sensor measurements during each pulse in order to provide such real time pulse control. The device may be configured to take such sensor measurements at any suitable frequency, e.g., approximately: every 250 μs, every 500 μs, every 1 ms, every 2 ms, every 5 ms, every 10 ms, every 20 ms, every 50 ms, every 100 ms, or any other suitable frequency.

Real time pulse control may be provided for treatments or configurations having a wide variety of pulse duration. For example, fractional treatment devices having a typical pulse duration of less than 20 milliseconds may provide real time pulse control to (a) control the duration or end time of each pulse and/or (b) provide one or more mid-pulse adjustments of one or more pulse parameters. Example fractional treatment devices having a typical pulse duration of between 1 ms and 10 ms may provide real time pulse control to (a) control the duration or end time of each pulse and/or (b) provide a series of mid-pulse adjustments (where appropriate) of one or more pulse parameters during each pulse, at any suitable mid-pulse adjustment frequency (e.g., approximately: every 500 μs, every 1 ms, every 2 ms, or any other suitable frequency). Example fractional treatment devices having a typical pulse duration of less than 1 ms may provide real time pulse control to (a) control the duration or end time of each pulse and/or (b) provide at least one mid-pulse adjustments (where appropriate) of one or more pulse parameters during each pulse, at any suitable mid-pulse adjustment frequency (e.g., approximately: every 500 μs, every 250 μs, or faster).

As another example, hair removal devices having a typical pulse duration of between 10 and 500 milliseconds may provide real time pulse control to (a) control the duration or end time of each pulse and/or (b) provide one or more mid-pulse adjustments of one or more pulse parameters. Example fractional treatment devices having a typical pulse duration of 100-500 ms may provide real time pulse control to (a) control the duration or end time of each pulse and/or (b) provide a series of mid-pulse adjustments (where appropriate) of one or more pulse parameters during each pulse, at any suitable mid-pulse adjustment frequency (e.g., approximately: every 10 ms, every 50 ms, every 100 ms, or any other suitable frequency). Example fractional treatment devices having a typical pulse duration of less than 100 ms may provide real time pulse control to (a) control the duration or end time of each pulse and/or (b) provide at least one mid-pulse adjustments (where appropriate) of one or more pulse parameters during each pulse, at any suitable mid-pulse adjustment frequency (e.g., approximately: every 5 ms, every 10 ms, every 20 ms, or any other suitable frequency).

In some embodiments, systems and methods disclosed herein may provide real time energy control to measure and adjust the amount of treatment energy applied to a treatment area so as to raise the superficial skin temperature by a specified amount. At least two treatment modes are considered: a gliding treatment mode and a stamping treatment mode. In the gliding treatment mode, the real time energy control may take into account the speed or displacement of the device gliding along the skin surface, as measured by a velocity sensor or a displacement sensor. If displacement occurs during a pulse, then the area exposed to light is larger than if the device is stationary, and the device may compensate for such movement by controlling the radiation source accordingly. In the stamping treatment mode, the treatment may be applied to one localized area of the skin at a time.

In some embodiments, measured diffuse-scattered light is used to calculate absorbed energy within tissue in real time (e.g., with a control response time less than 10 ms, less than 5 ms, less than 1 ms, or less than every 300 μs), in order to automatically control treatment energy in real time to deliver the desired amount of energy to accomplish a particular treatment objective. In some embodiments, such automatic real time control may avoid the need for active user input or control over treatment energy, such the device can be used efficiently and with good effect even by unskilled users. In some embodiments, the device may compact, inexpensive, and relatively easily manufacturable.

FIG. 2 illustrates an example dermatological treatment device 10 including a real-time energy control system 100, according to example embodiments. In this example embodiment, treatment device 10 may include a device body or housing 24 which houses or supports a laser 14, e.g., an 808 nm semiconductor laser, and a real-time energy control system 100, which includes a photodectector 102 (e.g., photodiode or phototransistor), one or more skin contact sensors 104, a displacement sensor 106, and any suitable control electronics 30 (e.g., memory devices, processor(s), and algorithms or other logic instructions) for analyzing signals from sensors 102, 104, and 106, and controlling the operation of laser 14 based on such analyzed signals. As discussed above, other embodiments may use any other suitable type of radiation source 14.

Laser 14 may be arranged to illuminate the skin surface 38 either in direct proximity or through a light guide 106, for example. Some light energy is absorbed by melanin in the epidermis 120 and a remaining portion is scattered in the volume of the underlying tissues 122. A portion of the scattered light impinges on one or more photodetectors 102, which may be arranged at a leading surface 110 of device body 24 in direct proximity to the skin, or coupled to the leading surface 110 via optics or a light pipe 112 as illustrated. Contact sensors 104 arranged around a perimeter of the device body 24 may be configured to determine whether the device is in good contact with the skin surface 38, to ensure that the light detected by photodectector 102 is substantially from subsurface scattering, rather than from surface reflection. Displacement sensor 106 may be configured to detect and monitor the displacement of device 10 along the skin during treatment.

Different skin types absorb light energy at a different rate, which is determined by the density of melanin in the epidermis. The light energy signal detected by photodectector 102 when the device 10 is in contact with the skin, relative to that detected while the device 10 is in contact with a white diffuser, is a measurement of the energy absorbed in the epidermis. Knowing the energy absorbed in the epidermis as a function of the translational distance along the skin, allows the calculation of the temperature rise in the epidermis, assuming a characteristic specific heat of skin. This permits device 10, and in particular real-time energy control system 100, to administer regulated doses of treatment energy (e.g., by controlling pulse parameters) that increases the epidermal temperature by a specified amount, independent of the melanin density in the treated skin area.

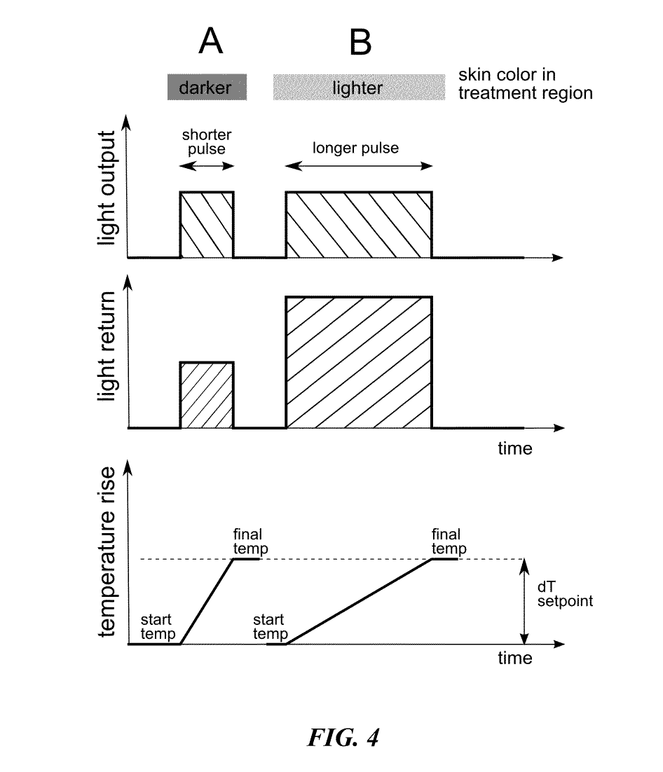

FIG. 4 illustrates components of an example real-time energy control system 100, according to one embodiment. As shown, real-time energy control system 100 may include an output light pipe 106 and a return light pipe 132 separated from each other by an opaque annular ring 130. Output light pipe 106 is configured to deliver treatment light from radiation source 14 to the skin, while return light pipe 132 is configured to collect and deliver to photodectector 102 a portion of the back-scattered treatment light from the skin. The opaque annular ring 130 between the two pipes 106 and 132 may act as a shield, blocking any direct transfer of light between pipes 106 and 132. In some embodiments, an air gap or low-index material, indicated at 140 and 142, may be provided immediately surrounding pipes 106 and/or 132 to promote total or near total internal reflection. The return light pipe 132 may have any suitable geometric features and surface texture to improve the uniformity of the detection sensitivity over its input area. With this configuration, the backscattered light may be averaged around the periphery of the treatment area, to better approximate the average value of the skin absorbed energy ratio (absorptance) in the treatment region, even if the skin melanin density is not spatially uniform. Although circular and cylindrical elements are illustrated in the example embodiment, any other suitable shapes may be used. For example, a rectangular light guide may be optically convenient for use with a high-aspect ratio radiation source, e.g., a laser bar.

In some embodiments, due to the level of optical treatment energy generated by device 10 (e.g., many watts), the photodetector 102 need not be very sensitive, and it may be practical to use a LED to detect the scattered return light, as an alternative to a photodiode or phototransistor. For example, an infrared LED was found to generate about 10 microamps signal with a 0.8 mm² input area exposed to the skin, which can be converted to a 1 V signal level with <20 microsecond rise time using an inexpensive op-amp with gain-bandwidth product of 1 MHz. A simple microcontroller can do an 8 or 10-bit A/D conversion and several floating-point operations in less than 200 microseconds. A simple current-controlled laser drive circuit can be turned off in less than 20 microseconds. Thus, the back-scattered energy can be measured, the absorbed energy calculated, and the output pulse length and hence treatment energy for a given area can be adjusted in less than 1 millisecond, less than 500 microseconds, or even less than 250 microseconds, thus enabling a flexible and responsive real-time energy control system 100.

The example systems and methods disclosed herein may be used to control a dermatological treatment light source 14 by measuring and adjusting the amount of energy applied to the treatment area so as to raise the superficial skin temperature by a specified amount. At least two operational modes are considered: a gliding treatment mode and a stamping mode. In the gliding mode, the real-time energy control system 100 may account for the distance travelled along the skin surface as determined based on measurements of displacement sensor 106 or a velocity sensor 26. If some displacement occurs during a pulse, then the area exposed to light is larger than if the device is stationary, and real-time energy control system 100 may apply suitable algorithms to compensate accordingly. In the stamping mode, the treatment may be applied to one localized area of the skin at a time.

### Theory of Operation for Certain Embodiments

FIG. 4 illustrates aspects of real-time energy control for two example skin types or pigmentation levels, indicated as “darker” and “lighter,” according to certain embodiments. More particularly, the figure illustrates how a device 10 may control a pulsed light source 14 for different skin types or regions having a different melanin density, and hence rate of energy absorption. In a relatively darker skin region (A) there is more light absorbed and less light returned. The level of returned light detected by photodetector 102 is compared with the calibration level and the light source 14 is cut off sooner. On a relatively lighter skin region (B) there is less light absorbed, and more light is returned, as detected by photodetector 102. Acting on the larger returned light signal, the real-time energy control system 100 allows the light pulse to continue for a longer time. The skin temperature rises more slowly but reaches the same point. In this example, the light power is kept constant and the pulse length is varied. In other embodiments, control system 100 may adjust the light power level, or both power level and duration in combination, to achieve similar results.

The temperature rise dT for a given skin treatment area, in the adiabatic approximation (the energy input duration is short compared with the thermal diffusion time) is given by:

dT=Q/C and equation 1

Q=Ab*Pin*t equation 2

where:

dT=increase in skin temperature (degrees C. or K)

Q=energy absorbed in skin (joules)

C=heat capacity of skin treatment area (joules/kelvin)

Ab=absorptance (ratio of light absorbed in skin to light incident on skin)

Pin=incident light power (watts)

t=time (seconds)

The returned light measured by the photodetector 102 is given by

Lrs=(k*Lin)/Ab or, equation 3

Ab=(k*Lin)/Lrs equation 4

where:
Lrs =light return sensor output (volts)
k=scaling factor (2* diffuse scattering ratio*total detector efficiency, volts/watt)
Lin =light input power (watts)

Ab =absorptance (ratio of light absorbed in skin to light incident on skin, or Lab/Lin)
note: Ab is 1.0 for a perfect black absorber, and 0 for a perfect scattering medium

By conservation of energy we have Lin=Lab+Ls (light input=light absorbed+light scattered) and hence Lin−Ls=Lab. So we can rewrite the definition Ab=Lab/Lin as Ab=Lab/(Lab+Ls). In the regime where much less light is absorbed than scattered (Lab<<Ls), to a good approximation Ab=Lab/Ls or in other words, the scattered light Ls is inversely proportional to the absorptance Ab. Finally, the detected value Lrs is proportional to total scattered light Ls, for some given scattering function and a fixed detector geometry and position relative to the illuminated skin volume.

Under the assumption that the terms inside the scale factor k remain constant (uniform diffuse scattering in tissue, and fixed detector geometry and coupling efficiency), the skin absorptance Ab can be determined directly from the measured parameter Lr and the previously determined calibration constant k and known source power Lin. This in turn enables the calculation of temperature dT after a given time interval t. The control system implemented on a microcontroller may then adjust the duration of the treatment light pulse or pulse train such that the skin temperature increase dT reaches the desired value.

### Example Implementations of Real-Time Energy Control

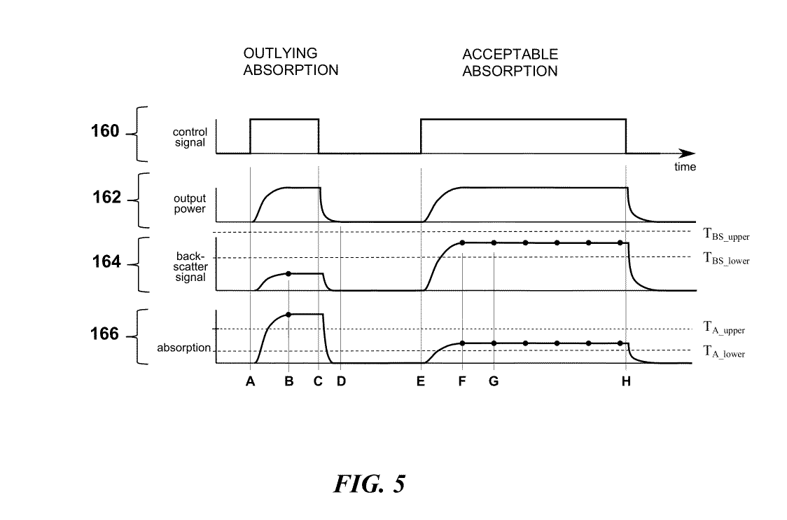

FIG. 5 illustrates example treatment control sequences implemented by a treatment device 10 including a real-time energy control system 100, according to an example embodiment. In particular, the figure shows (a) a graph 160 of the control signal implemented by real-time energy control system 100, (b) a graph 162 the resulting treatment radiation output power delivered by the device 10, (c) a graph 164 a corresponding back-scatter signal detected by photodetector 102, and (d) a graph 166 the energy absorption in the skin corresponding to the detected back-scatter signal, for each of (1) an example out-of-tolerance-limits energy absorption situation, indicated as “Excessive Absorption” (left side of each graph 160-166) and (2) an example within-tolerance-limits energy absorption situation, indicated as “Acceptable Absorption” (right side of each graph 160-166).

Turning first to the example “Outlying Absorption” situation (left side of each graph 160-166), at time (A) a control signal turns on the treatment energy source 14. This causes the output treatment power to rise to a defined level. After a power-on ramp-up and settling of the output power, the detected backscatter signal is sampled at time (B) (e.g.,) by photodetector 102 and passed to an A/D converter connected to a processor, e.g., a microcontroller or microprocessor. Sampling time (B) may be preset based on a predetermined delay time T_{D} after time (A) based on a known power-on rise time of the output power profile for the particular radiation source and/or current operating parameters.

The processor uses the sampled value to calculate the rate of energy absorption in the tissue, which is proportional to tissue absorptance and the treatment radiation output power. Note from Equation 4 that the energy absorption rate is inversely proportional to the backscatter signal. If the absorption value meets certain criteria, such as exceeding a preset upper threshold level (e.g., T_{A}_{_{—}}ᵤₚₚₑᵣ shown in FIG. 5), or being outside a preset tolerance window (e.g., outside of the window defined between T_{A}_{_{—}}ᵤₚₚₑᵣ and T_{A}_{_{—}}_{lower}), control system 100 may turn off the treatment energy source 14 at time (C) and after a power-off fall time has elapsed the output power reaches to a negligible value at time (D). Alternatively, control system 100 may continue the pulse but adjust one or more operational parameters of the radiation delivery in real time to bring the absorption back within the defined limits, e.g., below the upper threshold T_{A}_{_{—}}ᵤₚₚₑᵣ or within the threshold window between T_{A}_{_{—}}ᵤₚₚₑᵣ and T_{A}_{_{—}}_{lower}.

This mode of operation provides a safety check that may prevent a dangerous amount of total energy from being emitted, e.g., where the treatment window or aperture 44 is not in good contact with the skin, or the localized treatment area is too dark for safe treatment (for example due to strong pigmentation, or a tattoo). In some embodiments, taking into account all rise times, fall times, latencies and digital calculations, the time duration between the energy starting and stopping (time interval A-D in FIG. 5) is less than 2 milliseconds.

The duration from time (B) to time (C) may consist of or consist essentially of the time required for completion of the signal collection by photodetector 102, conversion to digital signals, and digital signal processing (e.g., digital calculations) by the processor, without significant delays unrelated to the automated completion of such operations, such that the detection of the threshold-exceeding absorption and shut-off of the radiation source 14 is provided in real time. In some embodiments, these operations (i.e., the duration from time (B) to time (C)) are performed in less than 1 millisecond. In particular embodiments, these operations are performed in less than 500 microseconds, or even less than 250 microseconds.

Turning now to the example “Outlying Absorption” situation (right side of each graph 160-166), the control signal implemented by control system 100 starts the treatment output power at time (E) and the backscatter return signal is sampled at time (F) (e.g., the predetermined delay time T_{D} after time E). In this case the return signal is above a threshold level indicating a safe treatment regime. Treatment proceeds, and the return signal is sampled at predetermined time intervals (G, etc.) until a predetermined total energy amount is deposited, which may be selected to cause a desired treatment effect in that treatment area. Once the predetermined total energy amount has been delivered, control system 100 turns of the radiation source 14 at time (H).

Control system 100 may calculate the time (H) for turning off the radiation source 14 in any suitable manner, e.g., (a) by calculating the time at which the predetermined total energy amount is delivered based on a single backscattered light measurement or corresponding absorption calculation (e.g., at time F), or (b) by calculating the time at which the predetermined total energy amount is delivered based on a calculated average of multiple backscattered light measurements or corresponding absorption calculations (e.g., at times F, G, etc.), or (c) by calculating the time at which the predetermined total energy amount is delivered based on a calculated sum of all backscattered light measurements or corresponding absorption calculations during the pulse, e.g., by integrating the area under the absorption curve to determine the total energy absorbed by the treatment region. Using the integrated energy method may be advantageous, e.g., if the treatment region is heterogeneous and the treatment aperture area has occupied more than one location during the treatment.

In some embodiments, control system 100 turns off the radiation source in response to determining that the predetermined total energy amount has been delivered. In such embodiments, control system 100 can control the treatment pulse duration can be controlled with a resolution limited only by data processing times and inherent latency of electronics of real-time energy control system 100, e.g., a resolution of 1 millisecond, 500 μs, 250 μs, etc.

In other embodiments, during the delivery of a pulse, control system 100 calculates (i.e., estimates) the future time at which the predetermined total energy amount will have been delivered for that pulse, e.g., based on one or more measurements of photodetector 102 that have been taken at that point during the pulse (e.g., using any of the algorithms and calculation methods discussed above). Further, control system 100 may account for an amount of energy to be delivered during the ramp-down of the output power, and thus calculate a future shut-off time of radiation source 14 that results in the predetermined total energy amount being delivered during the full pulse, including the ramp-down period. In other words, control system 100 may calculate the shut-off time by calculating (i.e., estimating) the future time at which the predetermined total energy amount will have been delivered for that pulse, and then applying a backwards time offset to account for a known (or estimated) energy delivery after initiating the shut-off of the radiation source. In these types of embodiments, which act predictively (rather than acting in response to determining that the predetermined total energy amount has been delivered), control system 100 is not limited by data processing times or system latency, and thus may provide an even better pulse duration resolution, e.g., within 500 μs, 250 μs, 100 μs, or even 10 μs, depending on the particular configuration or resolution demands for the particular treatment.

If an “Acceptable Absorption” situation moves becomes an “Outlying Absorption” situation mid-pulse, i.e., the calculated absorption moves beyond or outside of the preset threshold value(s) (e.g., T_{A}_{_{—}}ᵤₚₚₑᵣ and/or T_{A}_{_{—}}_{lower} shown in FIG. 5), control system 100 may either (a) interrupt or turn off the treatment energy source 14 or (b) continue the pulse but adjust one or more operational parameters of the radiation delivery in real time to bring the absorption back within the defined limits, e.g., below the upper threshold T_{A}_{_{—}}ᵤₚₚₑᵣ or within the threshold window between T_{A}_{_{—}}ᵤₚₚₑᵣ and T_{A}_{_{—}}_{lower}

As discussed above, in some embodiments, real-time energy control system 100 may automatically account for movement of the device 10 relative to the skin during the radiation delivery, e.g., during each pulse in a pulsed-radiation device. For example, if a pulsed-radiation device 10 is operated in a gliding mode, wherein the device is glided across the skin during the delivery of radiation pulses to the skin, real-time energy control system 100 may monitor the displacement of device based on signals from displacement sensor 106, a velocity sensor 26, or any other suitable sensor(s), and compensate for such displacement when calculating the backscattered light and/or corresponding light absorption during the radiation pulse, e.g., such that each treated location on the skin receives a predetermined amount of energy during a pulse, or such that no location on the skin receives more than a predetermined upper limit of energy during a pulse. Control system 100 may utilize any suitable algorithms for such compensation, e.g., based on the detected displacement or velocity of device 10, the detected back-scattered light, the size and geometry of the instantaneous treatment spot on the skin (which may be equal to or smaller than the size and geometry of the treatment aperture or window 44), and/or any other relevant information.

In some embodiments, instead of the treatment energy being delivered in a single pulse within a treatment region, control system 100 may generate a pulse train in which the treatment power is turned on and off more than once, with a fixed or variable frequency, and having a fixed or variable duty cycle. For example, a variable duty cycle pulse train could be used to rapidly adjust the output power, and hence rate of deposited treatment energy. This may provide control over the temperature profile as a function of time of the treated region.

Thus, in any of the manners discussed above, real-time energy control system 100 may provide “real time pulse control” for controlling the pulse duration of individual pulses based on one or more mid-pulse measurements of backscattered light, to thereby provide improved dosage control on a pulse-by-pulse basis, e.g., to deliver a desired amount of energy to accomplish a particular treatment objective. In some embodiments, such automatic real time control may avoid the need for active user input or control over treatment energy, such the device can be used efficiently and with good effect even by unskilled users. In some embodiments, the device may compact, inexpensive, and relatively easily manufacturable.

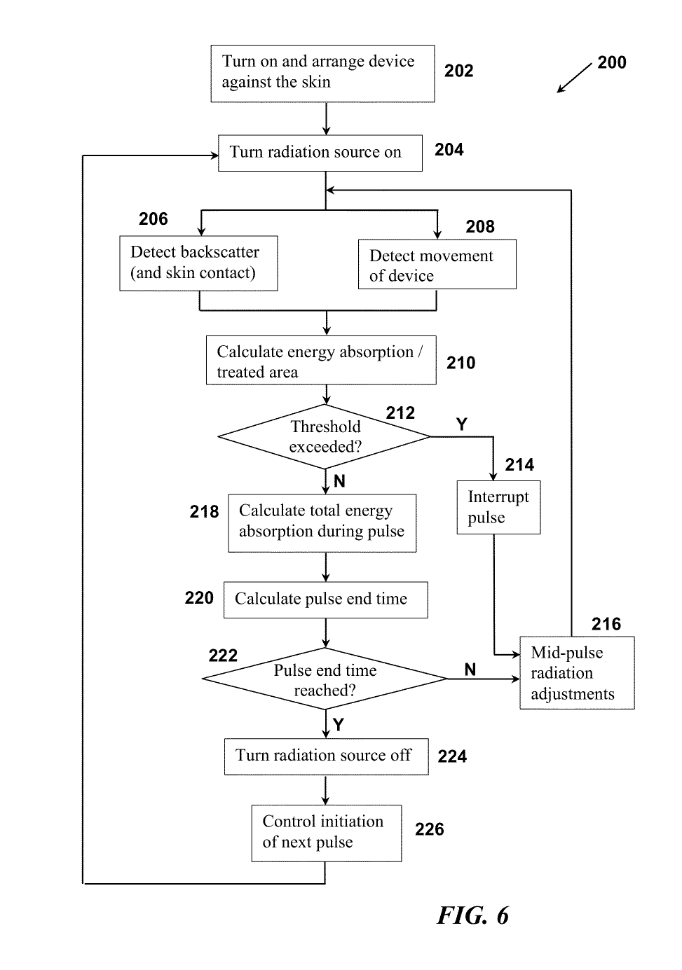

FIG. 6 illustrates an example method 200 of providing real-time energy control during a dermatological treatment 10, according to an example embodiment. At step 202, a user turns on device 10 and arranges the application end of device 10 against a target area of skin to begin a treatment session (or alternatively, device 10 may automatically turn itself on in response to detecting that the device has been placed against the skin, or upon some other triggering event).

At step 204, control system 100 initiates a treatment radiation pulse, e.g., based on a time-based control protocol or upon predefined triggering event(s) (e.g., contact detected by skin contact sensor(s) and/or minimum displacement or velocity of the device across the skin) This step may correspond to time (A) or (E) shown in FIG. 5.

At step 206, after a predetermined delay time e.g., based on a known or estimated output power rise time for the particular radiation source 14, photodetector 102 may detect backscattered light from the skin. In some embodiments, step 206 also includes detection of adequate skin contact via one or more skin contact sensors 104, e.g., to ensure the light detected by photodetector 102 is backscattered treatment light, e.g., as opposed to surface-reflected treatment light or ambient light. In some embodiments, control system 100 may automatically interrupt the pulse (by turning off radiation source 14) in response to signals indicating a lack of adequate skin contact. Step 206 may correspond to times (B), (F), (G), etc. shown in FIG. 5.

At step 208, a displacement sensor 106 or a velocity sensor 26 may detect the movement, if any, of the application end 110 of device 10 across the skin, e.g., movement associated with a gliding mode operation of device 10. The movement detection may be performed before, after, or partially or fully simultaneous with the backscattered-light detection of step 206, and each type of detection may be performed any suitable similar or different frequency.

At step 210, real-time energy control system 100 may calculate an energy absorption based on the backscattered-light and device movement (if any) detected as steps 206 and 208, by applying any suitable algorithm disclosed herein or otherwise within the level of knowhow of those skilled in the art. For example, if movement of device 10 is detected based on the measurement(s) at step 208, control system 100 may utilize any suitable algorithms to calculate the energy absorption per instantaneous illuminated area or instantaneous treatment spot within the overall treatment spot area traced during the pulse.

At step 212, control system 100 may determine whether the energy absorption calculated at step 210 is “acceptable” or “outlying,” e.g., by comparing the calculated absorption to one or more threshold values, such as T_{A}_{_{—}}ᵤₚₚₑᵣ and/or T_{A}_{_{—}}_{lower} threshold values discussed above with reference to FIG. 5. If the calculated absorption is determined to be “acceptable,” the method may proceed to step 218. However, if the calculated absorption is determined to be “outlying,” control system 100 may either (a) interrupt or turn off the treatment energy source 14, as indicated at step 214 or (b) continue the pulse but adjust one or more operational parameters of the radiation delivery in real time to bring the absorption back within the defined limits, e.g., below the upper threshold T_{A}_{_{—}}ᵤₚₚₑᵣ or within the threshold window between T_{A}_{_{—}}ᵤₚₚₑᵣ and T_{A}_{_{—}}_{lower}, as indicated at step 216.

At step 218, control system 100 may calculate a running total of the energy absorption of the skin for the pulse, based on one or a series of energy absorption calculations at step 210 (as the method may pass through multiple iterations of steps 206-212 during each pulse). Again, control system 100 may apply any suitable algorithm disclosed herein or otherwise within the level of knowhow of those skilled in the art. For example, may integrate a series of calculated absorption values as discussed above, to determine a running total of the energy absorption during the pulse. Also, if movement of device 10 is detected based on the measurement(s) at step 208, control system 100 may utilize any suitable algorithms to calculate the total energy absorption per instantaneous illuminated area or instantaneous treatment spot within the overall treatment spot area traced during the pulse.

At step 220, control system 100 may calculate an end time for the pulse, e.g., based on the running total of the energy absorption calculated at step 281, and according to any of the techniques disclosed above for calculating the pulse duration/end time.

At step 222, control system 100 may determine whether the pulse end time has been reached. If not, the pulse continues and the method may return to steps 206 and 208 for further light-backscatter and device-movement detections and resulting absorption calculations. In some embodiments, as shown in FIG. 6, control system 100 may make one or more mid-pulse adjustments to the radiation delivery, e.g., based on a calculated difference between the instantaneous energy absorption calculated at step 210 and a preset target absorption value (e.g., a preset value between T_{A}_{_{—}}ᵤₚₚₑᵣ and T_{A}_{_{—}}_{lower}) or based on any other target parameters or threshold values.

Alternatively, if control system 100 determines at step 222 that the pulse end time has been reached, control system 100 may turn off the radiation source 14 at step 224, and subsequently control the initiation of the next pulse at step 226, e.g., based on a present timing protocol, a minimum displacement of device 10 across the skin, and/or any other triggering events or conditions.

Although the disclosed embodiments are described in detail in the present disclosure, it should be understood that various changes, substitutions and alterations can be made to the embodiments without departing from their spirit and scope.

1. A dermatological treatment device, comprising:
a device body;
a radiation source housed by the device body and configured to emit pulses of treatment light for delivery to the skin to provide a dermatological treatment;
a photo detector housed by the device body and configured to detect light from the skin; and
control electronics housed by the device body and configured to:
during the delivery of a particular treatment light pulse from the radiation source, receive signals from the photo detector;
during the delivery of the particular treatment light pulse, automatically calculate at least one pulse parameter value for the particular treatment light pulse based at least on the signals received from the photo detector; and
automatically control at least one parameter of the particular treatment light pulse based on the at least one calculated pulse parameter value for the particular treatment light pulse.

2. The dermatological treatment device of claim 1, wherein the photo detector is configured to detect backscattered treatment light from the skin, the detected backscattered treatment light originating from the particular treatment light pulse.

3. The dermatological treatment device of claim 1, wherein the photo detector is configured to detect backscattered light from the skin, the detected backscattered treatment light originating from a radiation source other than the radiation source that provides the treatment light pulses.

4. The dermatological treatment device of claim 1, wherein automatically controlling at least one parameter of the particular treatment light pulse comprises automatically controlling the operation of the radiation source.

5. The dermatological treatment device of claim 1, wherein:
automatically calculating at least one pulse parameter value for the particular treatment light pulse comprises automatically calculating a pulse duration for the particular treatment light pulse; and
automatically controlling at least one parameter of the particular treatment light pulse based on the at least one calculated pulse parameter value for the particular treatment light pulse comprises automatically implementing the calculated pulse duration for the particular treatment light pulse.

6. The dermatological treatment device of claim 1, wherein:
automatically calculating at least one pulse parameter value for the particular treatment light pulse comprises automatically calculating a power level of the radiation source for the particular treatment light pulse; and
automatically controlling at least one parameter of the particular treatment light pulse based on the at least one calculated pulse parameter value for the particular treatment light pulse comprises automatically adjusting the power level of the radiation source during the delivery of the particular treatment light pulse based on the calculated power level.

7. The dermatological treatment device of claim 1, wherein:
the photo detector is configured to make multiple measurements of detected light during the particular treatment light pulse; and
the control electronics are configured to calculate the pulse duration for the particular treatment light pulse based at least on the multiple measurements by the photo detector.

8. The dermatological treatment device of claim 1, wherein automatically calculating at least one pulse parameter value for the particular treatment light pulse based at least on the signals received from the photo detector comprises:
calculating an energy delivered to the skin based on the signals received from the photo detector during the particular treatment light pulse; and
calculating at least one of a pulse duration and a power level for the particular treatment light pulse based at least on the calculated energy delivered to the skin.

9. The dermatological treatment device of claim 1, wherein automatically calculating at least one pulse parameter value for the particular treatment light pulse based at least on the signals received from the photo detector comprises:
calculating an energy delivered to the skin based on the signals received from the photo detector during the particular treatment light pulse;
calculating a skin temperature or change in skin temperature based on the calculated energy delivered to the skin; and
calculating at least one of a pulse duration and a power level for the particular treatment light pulse based at least on the calculated skin temperature or change in skin temperature.

10. The dermatological treatment device of claim 1, wherein the device body comprises a portable, hand-held body.

11. A method of automated control of a dermatological treatment device, comprising:
operating a radiation source to emit treatment light pulses for delivery to the skin to provide a dermatological treatment;
detecting light from the skin using a photo detector;
during the delivery of a particular treatment light pulse from the radiation source, receiving signals from the photo detector;
during the delivery of the particular treatment light pulse, automatically calculating at least one pulse parameter value for the particular treatment light pulse based at least on the signals received from the photo detector; and
automatically controlling at least one parameter of the particular treatment light pulse based on the at least one calculated pulse parameter value for the particular treatment light pulse.
